(19) United States
(12) Patent Application Publication
(10) Pub. No.: US 2020/0246037 A1
(43) Pub. Date: Aug. 6, 2020

(54) Optic scalpel

(71) Applicant: Joshua Tyson, New York, NY (US)

(72) Inventor: Joshua Tyson, New York, NY (US)

(21) Appl. No.: 16/350,933

(22) Filed: Feb. 1, 2019

(51) Int. Cl. A61B 17/3211; A61B 90/30

(52) U.S. Cl. CPC A61B 17/3211; A61B 2017/00734; A61B 2090/309; A61B 90/30

(57) ABSTRACT

The optic scalpel is a scalpel available in various sizes and shapes that have circular led lights surrounding the base of the knife blade. When activated the lights provide additional lighting for surgical procedures. The optic scalpel optimizes lighting conditions for accurate incisions or dissections. The optic scalpel improves from its predecessor by intergrading led lights with a scalpel. The optic scalpel can be used independently to illuminate a local area for incisions or tissue cutting. The optic scalpel is powered by cell batteries that are located in the handle. The led lights are activated by a switch on the top of the handle.

The optic scalpel is a surgical knife that has led lights surrounding the knife base. This invention the optic scalpel is designed to assist surgeons and medical personal during surgical procedures by illuminating a local area. The optic scalpel knife is designed and available in different sizes and shapes The optic scalpel can be used with it's own lighting independently for incisions and cutting soft tissue or simultaneously with hospital lights for surgical procedures. The lighting mechanism located in the handle of the optic scalpel is powered by cell batteries. The optic scalpel has a plurality of circular led lights surrounding the base of the knife blade that is activated by a switch on top of the handle. While using one hand to hold the optic scalpel, the thumb can be used to slide the switch forward for light activation and the switch is pushed back in place for light disbandment. When utilizing the optic scalpel for surgical procedures it minimizes shadow obstructions from the surgeon's hands or head. The optic scalpel is very effective in optimizing visibility in surgical incisions and anatomical dissections. The optic scalpel modernizes the use of the traditional scalpel by introducing led lights around the base of the knife blade that optimizes clarity and visibility for surgeons and medical teams. The optic scalpel can be used in arts and crafts for minute and precise cutting.

### BACKGROUND

Surgical lighting has always been critical for performing accurate medical procedures. The optic scalpel enables surgeons and medical teams with additional lighting for minute and precise incisions. The optical scalpel reduces eye fatigue in surgeons and medical teams by illuminating and reducing shadow obstructions. The optic scalpel is a surgical scalpel with led lights surrounding the base of the knife blade. When activated the led lights produce direct light output to the designated local area improving visibility during operations. The optic scalpel is an additional light source that helps alleviate shadows and optimizes lighting and visibility for surgeons and medical teams. The optic scalpel can be utilized independently while illuminating local areas for minor surgical procedures. The optic scalpel minimizes glare and shadows allowing doctors and medical personal to focus with optimum visibility on performing incisions or tissue removal. A secondary use for the optic scalpel is that it can be utilized as a hobby knife for arts and crafts.

### SUMMARY

Historically scalpels have been a relevant cutting instrument for performing surgical procedures and dissecting tissue. Scalpel blades are available in different sizes and shapes. The traditional surgical scalpel is a cutting instrument only. In contrast the optic scalpel is an improvement to the traditional scalpel by featuring attached led lights that illuminate and assist in visibility for surgical procedures. When activated lighting from the optic scalpel highlights the designated local area for incision or dissecting. The optic scalpel is distinguished from its predecessor by having circular led lights attached to the foremost tip of the handle. The optic scalpel is activated by a switch on top of the handle. The thumb is used to slide the switch forward for activation and back in place for light disbandment.

### BRIEF DESCRIPTION OF DRAWINGS

FIG. 1 Drawing shows three dimensional view of the optic scalpel knife with partial view of led lights 2, around the base of the blade.

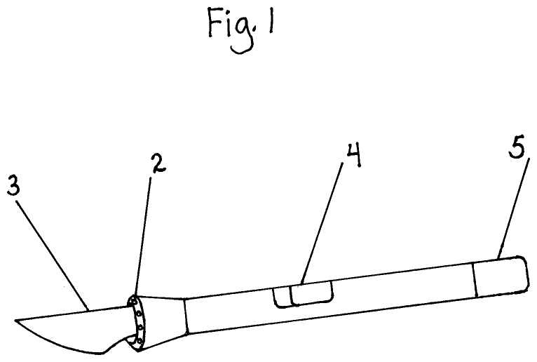

FIG. 2 This drawing is a three dimensional view of the optic scalpel showing activated led lights 2. The dots 1, protruding from the led lights around the blade represent light emissions from the led lights 2.

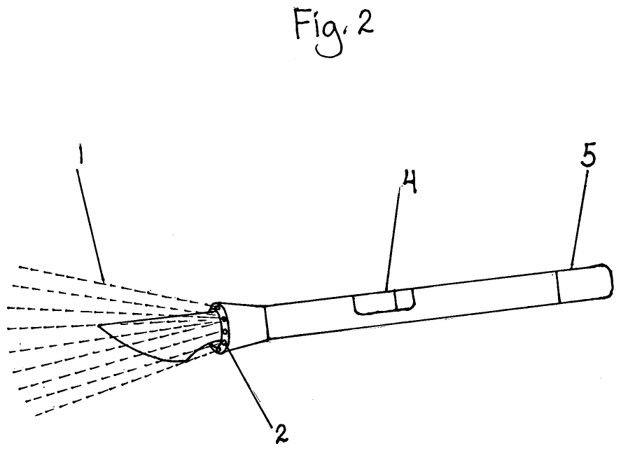

FIG. 3 Shows a three dimensional view of an angular shaped optic scalpel with led lights around the base of the blade.

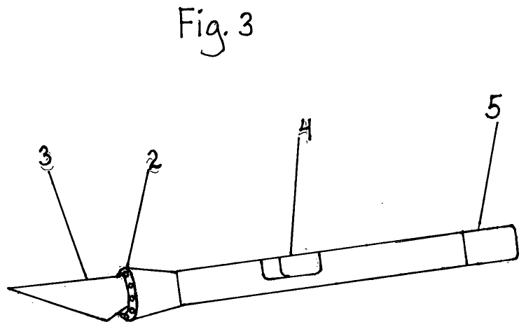

FIG. 4 Drawing shows a three dimensional view of an angular shaped optic scalpel knife with activated led lights 2. The dots 1, around the base of the blade represent light emissions from led lights 2. This drawing also shows the activation switch 4, pushed forward.

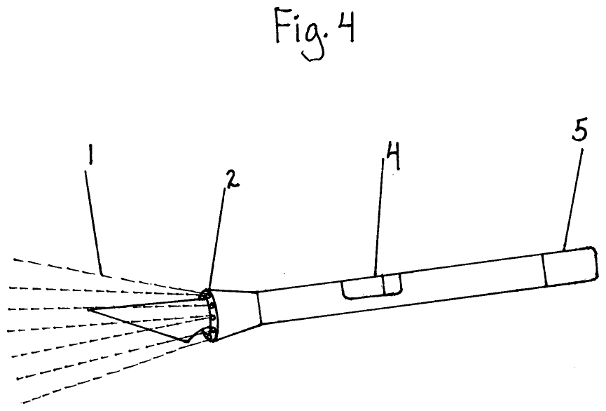

FIG. 5 This drawing shows the top view of the configuration of led lights encased in in handle tip frame around the optic scalpel blade.

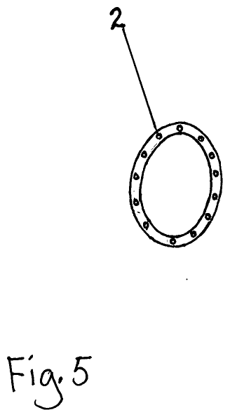

FIG. 6 Is a three dimensional view of the optic scalpel showing the bottom tip 5, separated from the central handle. The bottom is screwed off for cell battery insertion or removal.

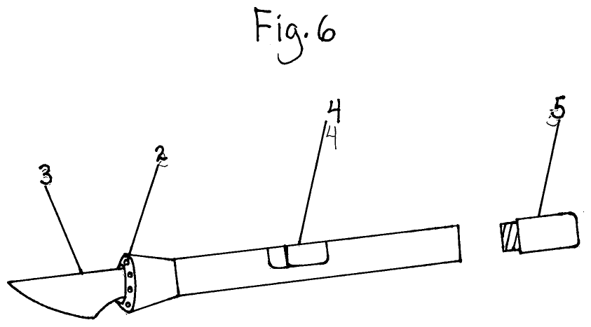

### DETAILED DESCRIPTION OF THE EMBODIMENTS OF THE INVENTION

Below please see reference numbers used throughout the description to refer to elements in the drawings:

1 Light emissions

2 lights around the base

3 Knife Blade

4 Light Switch

5 Bottom end of Handle

1. Surgical scalpel knife, comprising:
a knife blade wherein a plurality of lights are deposed around the base of the blade to configured to radially emit light around the knife blade.
a handle having at least one removable segment
a handle with a top activation switch
the knife activation switch is pushed forward for light activation and pushed back in opposite direction for light de-activation

2. the knife of claim 1 handle comprises a removable bottom end

3. the knife of claim 2, wherein the bottom end of the handle is configured to receive a power source
a surgical scalpel knife comprising:
a knife blade
a knife handle
a plurality of lights around the scalpel blade
a handle with at least one removable segment
a handle with a top light for
a plurality of lights
a handle with at least one removable segment, a removable bottom end and a removable body
wherein an activation switch disposed on the top of the handle is configured to radially emit light around the blade
wherein a plurality of lights are disposed around the blade base, configured to radially emit light around the blade
wherein the bottom end of the handle is configured to receive a power source
